(19) United States
(12) Patent Application Publication
(10) Pub. No.: US 2022/0062453 A1
(43) Pub. Date: Mar. 3, 2022

(54) UV WALK-THROUGH SANITIZER

(71) Applicant: CLAYTON SIGNS, Lake City, GA (US)

(72) Inventor: Andrew WILLIS, Senoia, GA (US)

(73) Assignee: CLAYTON SIGNS, Lake City, GA (US)

(21) Appl. No.: 17/463,183

(22) Filed: Aug. 31, 2021

Related U.S. Application Data
(60) Provisional application No. 63/073,266, filed on Sep. 1, 2020.

(51) Int. Cl. A61L 2/00

(52) U.S. Cl. CPC A61L 2/0047; A61L 2202/11

(57) ABSTRACT

A walk-through sanitizing device for sanitizing users with ultraviolet light as they walk through the sanitizing device. The sanitizing device includes a frame and at least one ultraviolet light source capable of killing or inactivating microorganisms.

### CROSS-REFERENCE TO RELATED APPLICATION

This application claims the priority benefit of U.S. Provisional Patent Application Ser. No. 63/073,266, filed Sep. 1, 2020.

### TECHNICAL FIELD

The present invention relates generally to the field of sanitizing devices, and more particularly to sanitizing stations utilizing ultraviolet (UV) germicidal irradiation.

### BACKGROUND

The spread of many pathogens, such as for example coronaviruses, Neisseria meningitidis, and Mycobacterium tuberculosis, can be largely reduced or prevented with proper and frequent sanitization practices. However, many sanitization processes require access to sanitizing materials which can be extremely limited during outbreaks and pandemics. Furthermore, many sanitization processes rely heavily on individuals' dedication and effort to ensure that proper steps are taken. Accordingly, needs exist for a sanitizing device providing a more convenient and automated decontamination process requiring no direct individual reliance or dependence on limited resources. It is to the provision of a walk-through ultraviolet (UV) sanitizer meeting these and other needs that the present invention is primarily directed.

### SUMMARY

In a first example form, the present invention provides a walk-through sanitizing device for sanitizing or decontaminating users. The walk-through sanitizing device includes a frame and one or more sanitizing dischargers. The one or more sanitizing dischargers are positioned along a passageway provided through the device frame.

In one aspect, the sanitizing dischargers emit ultraviolet light having a wavelength between about 200-280 nm, preferably between about 220-260 nm, and more preferably about 222 nm.

Optionally, the walk-through sanitizing device may include a thermal imaging camera and a digital display. Information captured by the thermal imaging camera may be transmitted to the digital display providing thermal mapping images of users who walk through the walk-through sanitizing device.

In another example form, the present invention relates to a sanitization station for sanitizing a user as the user walks through the sanitization station. The sanitization station includes a frame having a first side wall, a second side wall, and a top wall. The first side wall has a first inner surface; the second side wall has a second inner surface; and the top wall has a top inner surface. The first, second, and top inner surfaces define a passageway through the frame and are affixed with one or more ultraviolet light sources.

In one aspect, the one or more ultraviolet light sources emit ultraviolet light capable of inactivating microorganisms. More specifically, the one or more ultraviolet light sources emit ultraviolet light having a wavelength between about 200-280 nm, preferably between about 220-260 nm, and more preferably about 222 nm.

In another aspect, the sanitization station may further include a camera capable of capturing thermal imaging. Further, data of the thermal imaging may be transferred to a display providing visual representation of the data.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 is a perspective view of a walk-thru sanitizing station according to an example embodiment of the present invention.

FIG. 2 is a front elevation view of the sanitizing station of FIG. 1.

FIG. 3 is a side elevation view of the sanitizing station of FIG. 1.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

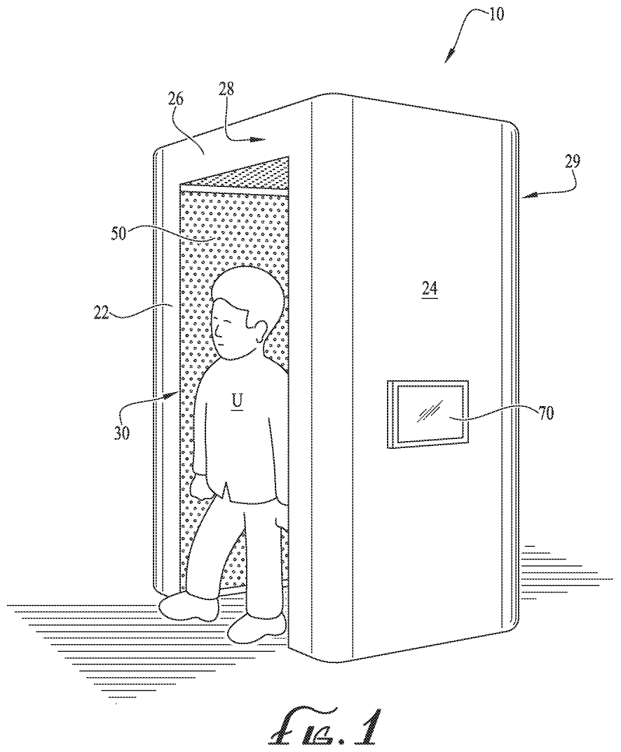

With reference now to the drawing figures, wherein like reference numbers represent corresponding parts throughout the several views, FIGS. 1-3 show a sanitizing device or station 10 according to an example embodiment of the present invention. The sanitizing station 10 allows a user U to be disinfected, sanitized and/or decontaminated, as he/she walks or otherwise travels, through the station. Typically, a user enters the station 10 through an entrance end 29 and leaves the station through an exit end 28. As the user travels between the entrance 29 and exit 28, he/she is disinfected, for example, by ultraviolet germicidal irradiation. The sanitizing station 10 is preferably deployed at the entrance or exit of a building or area, but it will be appreciated by those in the art that the sanitizing station may be placed or located as needed or suitable otherwise.

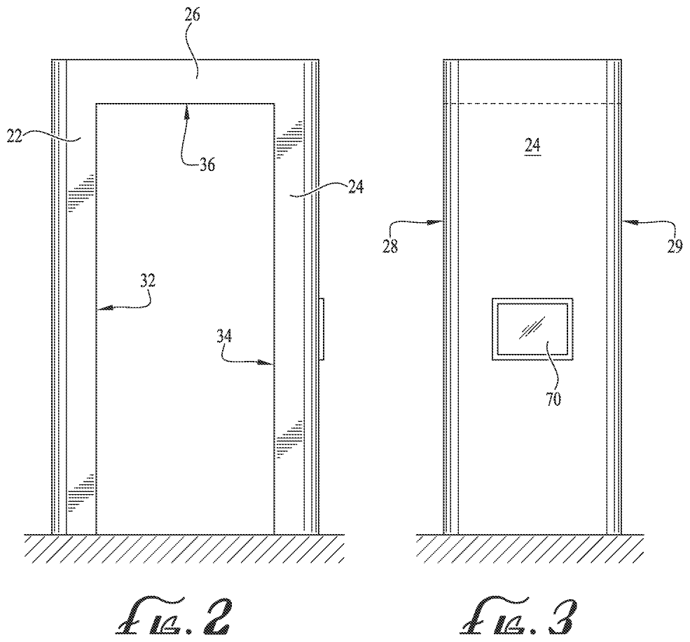

As shown in FIG. 2, the sanitizing station 10 generally comprises a first side wall or portion 22, second side wall or portion 24, and a top wall or portion 26. The first side, second side, and top walls form a frame over a ground or walking surface and defines a passageway or portal 30 through which the user travels. The passageway 30 is further defined by a first inner side surface 32, a second inner side surface 34, and a top inner surface 36 of the first side, second side, and top walls, respectively.

The inner surfaces 32, 34 and 36 of the passageway 30 are furnished with one or more dischargers which emit radiation and/or dispense sanitizing substance(s). In example embodiments, the sanitizing station 10 comprises one or more light sources 50, such as for example low-pressure mercury lamps, excimer lamp or far-UVC lamps, pulsed xenon lamps, and/or light-emitting diodes (LEDs), which emit short-wavelength ultraviolet light, such as for example UV-C, capable of killing or inactivating microorganisms. In example embodiments of the present invention, the one or more light sources 50 emit light in the range of 200-222 nm, or more preferably about 222 nm. In example embodiments, the UV light sources 50 are provided along the inner surfaces 32, 34 and 36 of passageway 30. The light sources 50 are positioned and oriented along each of the inner surfaces to maximize the exposure of UV light on the users as they travel through the passageway.

The sanitizing station 10 may also comprise compartments or shelves for storing and/or holding other sanitizing mediums or instruments, such as for example, portable and stationary hand sanitizer dispensers. The compartments or shelves may be integrated or removably attached to the walk-through sanitizing station 10. In some embodiments, the compartments or shelves may be attached or placed along the inner surfaces 32, 34 and 36 of the passageway 30. In other example embodiments, the compartments or shelves may be attached or placed along exterior surfaces of the walk-through sanitizing station, or preferably along the exterior surfaces of the walk-through sanitizing station adjacent to the entrance 29 and/or exit 28.

In another example embodiment, the sanitizing station comprises one or more cameras (not shown) and/or display screens 70. The cameras may be attached to the sanitizing station or placed remotely away from the sanitizing station. In example embodiments, the cameras are configured to capture audio, images or video of the passageway and the users traveling therethrough. The display screens are generally configured to display information captured by the cameras. In other example embodiments, the sanitizing station may include one or more thermographic cameras to capture thermographic data of users traveling through the sanitizing station. The thermographic data is then displayed on the display screens. In some example embodiments, the captured audio, image and or video information are saved to local and/or remote hard drives and/or servers for recollection as needed. In still other example embodiments, the display screens provide information and messages, such as for example weather, notices and news, to the users.

In example embodiments, the sanitizing station is powered from an external power source, such as for example, a building's central electrical system or an external power storage unit or generator. In other example embodiments, the sanitizing station is configured to receive and utilize one or more batteries.

In example modes of use, the sanitizing station 10 is provided to sanitize, or otherwise decontaminate, users as they travel therethrough. In example modes of use, the sanitizing stations are placed for example at the entrance of buildings and other facilities. As users U travel through the passage 30 of the sanitizing station 10, the users U and other objects passed through the passage are exposed to the ultraviolet light emitted from the plurality of LEDs surrounding the passage. In effect, the users U and other objects passed through the sanitizing station 10 are sanitized by ultraviolet germicidal irradiation.

While the invention has been described with reference to example embodiments, it will be understood by those skilled in the art that a variety of modifications, additions and deletions are within the scope of the invention, as defined by the following claims.

What is claimed is:

1. A walk-through sanitizing device for sanitizing users, the walk-through sanitizing device comprising a frame and one or more sanitizing dischargers.

2. The walk-through sanitizing device of claim 1, further comprising a passageway configured for users to travel therethrough and wherein the sanitizing dischargers are positioned along the passageway.

3. The walk-through sanitizing device of claim 2, wherein the sanitizing dischargers emit ultraviolet light.

4. The walk-through sanitizing device of claim 3, wherein the ultraviolet light has a wavelength between 200 nm and 222 nm.

5. The walk-through sanitizing device of claim 3, wherein the ultraviolet light has a wavelength of 222 nm.

6. The walk-through sanitizing device of claim 1, further comprising one or more cameras.

7. The walk-through sanitizing device of claim 6, wherein the one or more cameras are capable of capturing thermal images.

8. The walk-through sanitizing device of claim 6, furthering comprising at least one display.

9. The walk-through sanitizing device of claim 8, wherein images captured by the one or more cameras are transmitted and displayed on the at least one display.

10. The walk-through sanitizing device of claim 1, further comprising one or more storage compartments or dispensers.

11. The walk-through sanitizing device of claim 10, wherein the storage compartments or dispensers are adapted to store and dispense sanitizing liquids, gels, foams or towels.

12. A sanitizing station for sanitizing a user as the user walks therethrough, the sanitizing station comprising:
a first side wall with having a first inner surface,
a second side wall having a second inner surface, and
a top wall having a top inner surface;
wherein the first side wall, second side wall, and top wall define a frame and the first inner surface, second inner surface, and top inner surface define a passageway through the frame,
wherein at least one of the first, second, and top inner surfaces are lined with at least one ultraviolet light source.

13. The sanitizing station of claim 12, further comprising a camera.

14. The sanitizing station of claim 13, wherein the camera captures thermal images and the thermal images are transmitted to a digital display attached to the frame.

15. The sanitizing station of claim 14, further comprising at least one shelf positioned along the passageway.

16. The sanitizing station of claim 15, wherein the at least one shelf is adapted for holding portable sanitizer dispensers.

17. A method of sanitizing a user, the method comprising:
the user entering a walk-through sanitizing station;
exposing the user to ultraviolet light as the user traverses through the walk-through sanitizing station; and
the user exiting the walk-through sanitizing station.

18. The method of sanitizing a user of claim 17, wherein the walk-through sanitizing station comprises a left wall portion, a right wall portion, and a top wall portion, the left, right, and top wall portions defining a passageway.

19. The method of sanitizing a user of claim 18, wherein the walk-through sanitizing station further comprises one or more light sources secured along the passageway.

20. The method of sanitizing a user of claim 19, wherein the one or more light sources emit ultraviolet light having wavelength between 200 nm and 222 nm, inclusively.
